(19) United States
(12) Patent Application Publication
(10) Pub. No.: US 2016/0361190 A1
(43) Pub. Date: Dec. 15, 2016

(54) UNDERGARMENT

(71) Applicant: JKL IP Company LLC, Birmingham, MI (US)

(72) Inventors: Jodi E. Caden, Bloomfield Hills, MI (US); Burcu Bicak, Blacksburg, VA (US)

(21) Appl. No.: 14/739,564

(22) Filed: Jun. 15, 2015

(51) Int. Cl. A61F 5/03; A41B 9/00; A61F 13/14; A61F 5/02; A61F 13/00

(52) U.S. Cl. CPC A61F 5/03; A61F 5/028; A61F 13/00063; A61F 13/148; A41B 9/001

(57) ABSTRACT

The disclosure contemplates an undergarment comprising a torso portion and a bottom portion. The torso portion is to be worn around a torso of a wearer. The torso portion comprises one or more compression features. The bottom portion comprises a medicament area. The one or more compression features provide compression against at least a portion of the torso of the wearer. The medicament area interacts with a scar or wound and facilities healing thereof.

### TECHNICAL FIELD

The present teachings relate generally to apparel, and more particularly to an undergarment providing compressive support and facilitating recovery after a surgery or procedure.

### BACKGROUND

During pregnancy, a woman's body may go through various changes and stresses. For example, the woman's skin around the belly area may stretch and leave marks or lines after pregnancy (i.e. stretch marks). A woman may also realize weight gain, sagging skin, and/or a dimpled or lumpy appearance in other areas of her body, such as on her buttocks, hips, and thighs. During childbirth, some women undergo a cesarean section, which typically results in a post-cesarean scar approximately four to eight inches in length in the woman's belly area.

After a pregnancy, some women seek to reduce the appearance of the sagging skin, dimples lumps, and/or post-cesarean scar by using non-invasive treatments like wearing compression garments and/or applying various medicaments. However, some compression garments can rub against the skin causing irritation, which may be uncomfortable and undesirably prolong the healing and recovery process. Other garments may rub against one or more ancillary medicaments applied to a wound, scar or other area of the body, which may cause pain and/or cause the medicaments to move and/or dissipate.

There is thus a need for addressing or ameliorating one of more of the aforementioned disadvantages. For example, it would be desirable to also have an undergarment that can be used for treating weight gain, stretch marks, sagging skin, and/or dimpled or lumpy appearances in various areas of the body. It would be attractive to have an undergarment that includes one or more medicament areas facilitating treatment and/or healing of various wounds and scars. It would be desirable to have a single undergarment that can be used to treat stretch marks, sagging skin, weight gain, while also facilitating treatment and/or healing of wounds and scars. It would be desirable to have an undergarment that is lightweight, comfortable, supportive, and can be mass-produced relatively inexpensively.

### SUMMARY

It is thus among the objects of the disclosure to provide an undergarment for treating weight gain, stretch marks, sagging skin, and/or dimpled or lumpy appearances in one or more areas of the body, while also providing one or more medicament areas facilitating treatment and/or healing of wounds or scars and also reduction and/or elimination of odor. Moreover, the undergarment of the present disclosure is generally lightweight, comfortable, supportive, durable, and can be mass-produced relatively inexpensively.

The disclosure also provides an undergarment comprising a torso portion and a bottom portion. The torso portion can be worn around a torso of a wearer. The torso portion comprises one or more compression features. The one or more compression features provide compression against at least a portion of the torso of the wearer. The bottom portion comprises a medicament area. The medicament area interacts with a scar or wound and facilities healing thereof.

The disclosure further provides an undergarment, comprising a compression band, a plurality of compression strips, and a medicament area comprising at least some silver. The compression band and the plurality of compression strips provide compression against at least a portion of a torso of a wearer. The silver interacts with a scar or wound on the torso of the wearer and facilities healing thereof and reduction and/or elimination of odor.

Further yet, the present disclosure provides an undergarment comprising a torso portion and a bottom portion. The torso portion can be worn around a torso of a user. The torso portion comprises a compression material, a compression band, and a plurality of compression strips. The compression material, the compression band, and the plurality of compression strips provide compression against at least a portion of the torso of the wearer. The bottom portion is worn around at least a portion of a buttocks of a user. The bottom portion comprises a medicament area. The medicament area includes at least some silver that interacts with a scar or wound at a cesarean incision site and facilities healing thereof. The undergarment may be substantially free of any torso portion and may instead include only a bottom portion.

### DESCRIPTION OF THE DRAWINGS

FIG. 1 illustrates a front view of an undergarment according to the present teachings.

FIG. 2 illustrates a front view of the undergarment according to the present teachings.

FIG. 3 illustrates a back view of the undergarment according to the present teachings.

FIG. 4 illustrates a front view of the inner surface of the undergarment according to the present teachings.

### DETAILED DESCRIPTION

The teachings herein make advantageous use of apparel. Specifically, the present teachings disclose an undergarment. The undergarment can be worn by any person, of any age. It is envisioned that the undergarment can worn in lieu of regular undergarments and/or underwear. The undergarment can be comfortably worn under any clothing. In some instances, it is envisioned that the undergarment can be worn in everyday use. The undergarment can be worn around the torso, hips, buttocks, and/or thighs of any person. Preferably, the undergarment is a flexible garment with sufficient elasticity to stretch over parts of the wearer's body. The undergarment may be of sufficient dimensions to provide coverage of a portion or all of a wearer's torso, such as a belly; an area below the belly, such as the wearer's bottom, waist, hips, buttocks, portion of his or her legs; or combination thereof. Preferably, the undergarment may be expanded and stretched to be firmly but comfortably worn around the torso of a wearer.

The undergarment may be offered to a person in varying sizes (e.g., sizes designated by Small, Medium, Large, etc., or based on a person's specific measurements), which may be helpful as the person's body changes (e.g., as the person loses or gains weight). The undergarment may have a width that is at least about 5 inches. The undergarment may have a width that is less than about 20 inches. The undergarment may have a width that is at least about 9 inches. The undergarment may have a width that is less than about 15 inches. The undergarment may have a length that is at least about 5 inches. The undergarment may have a length that is less than about 25 inches. The undergarment may have a length that is at least about 9 inches. The undergarment may have a length that is less than about 18 inches.

The size of the medicament area may also vary depending on the purpose and size of the undergarment. The medicament area may extend to a width of at least about 2 inches at its widest point. The medicament area may extend to a width of less than about 15 inches at its widest point. The medicament area may extend to a length of at least about 2 inches at its widest point. The medicament area may extend to a width of less than about 12 inches at its widest point. The medicament area may extend to a length of at least about 4 inches at its widest point. The medicament area may extend to a width of less than about 9 inches at its widest point. The medicament area may extend to a width of at least about 5 inches at its widest point. The medicament area may extend to a width of at least about 10 inches at its widest point. The medicament area may extend toward the back of the undergarment to contact at least a portion of the buttocks. Thus the length of the medicament area may extend even longer, such as to at least about 5 inches, at least about 9 inches, at least about 15 inches or even at least about 20 inches.

It is envisioned that the undergarment may also have one or more straps to attach to a woman's bra or to be worn on a wearer's shoulders or around his or her neck to provide additional support to the weight of the wearer's belly and back and to assist in holding the garment in the proper position. These straps may be adjustable in length, removable, repositionable, convertible, or combination thereof. The undergarment, as described herein, contemplates different features, all of which, alone or in combination, are within the scope of the teachings.

The undergarment can be worn by a person after a surgery or procedure. The undergarment may reduce scarring, swelling, sensitivity, pain, itching, and discomfort resulting from a surgery or procedure. The undergarment can be worn by a person undergoing bodily changes such as weight loss or weight gain; a person desiring additional support and/or compression for his or her body; a woman after giving birth, whether vaginal or cesarean, and the like. The undergarment may be worn post-pregnancy for slight compression and smoothing. The undergarment may include features and/or areas that may facilitate treatment, rehabilitation, and recovery of wounds and/or scars after any surgery or procedure, such as a vaginal child birth or caesarian section.

One or more portions of the undergarment (i.e., a torso portion, a bottom portion, or both) may be fabricated from any suitable material for performing one or more of the functions disclosed herein. For example, one or more portions of the undergarment may comprise a knit material; a woven material; or a non-woven material. One or more portions of the undergarment may be made of a breathable fabric, stretchable fabric, compression fabric, flexible fabric, elastic fabric, or combination thereof. One or more portions of the undergarment may comprise any combination of a polyester, a polyamide such as nylon, an elastane/spandex, cotton, bamboo, modal, rayon, lyocell such as Tencel, other man-made materials, the like, or combination thereof. One or more portions of the undergarment may be a breathable knit fabric, such as nylon or spandex (e.g., Lycra®). One or more portions of the undergarment may be a nylon/Lycra® spandex blend. Benefits of the fabric disclosed herein include, but are not limited to, being easy to clean and being resistant to fungi, molds, mildew, odors and chemicals.

One or more portions of the undergarment (i.e., the torso portion, the bottom portion, or both) may comprise a moisture locking material. This moisture locking material may be a lining in the inner surface of the undergarment and may contact the skin to confine skin products, medicaments, etc. to the torso or the desired areas of the skin of the anatomy. The moisture locking material may be part of or integrated into the knit fabric. The moisture locking material may also keep external moisture out and away from the covered skin of the wearer. The moisture locking components, such as nylon, polyester, and other fibers such as rayon, bamboo, Tencel, and the like, may allow moisturizers, medicaments, or other products to seep into the skin as opposed to being absorbed by the fabric. Moisture locking components also allow one or more portions of the undergarment to be easily cleaned.

One or more portions of the undergarment (i.e., the torso portion, the bottom portion, or both) may comprise a moisture wicking material to pull moisture such as sweat and perspiration away from the wearer's skin. This may help keep the person cool in hot or humid conditions, or it may help keep the person warm in cool temperatures. A composition of about 20% or more of polyester, nylon, or any yarns with wicking properties, including derivative yarns made from a blend of polyester and/or nylon, or any yarns treated to have wicking properties may be used to achieve results with or without spandex. One or more portions of the undergarment may also have quick drying capabilities. The quick dry components, such as nylon or other fiber contents, allow the person to feel comfortable wearing the undergarment all day with moisturizers, creams, oils, and the like, remaining in place.

One or more portions of the undergarment (i.e., the torso portion, the bottom portion, or both) may be seamless. One or more portions of the undergarment may have one or more seams. The undergarment may include one or more hems (e.g., a hem at the torso opening and/or at the one or more leg openings to reduce the risk of the edges of the garment fraying). The undergarment may comprise a single material. One or more portions of the undergarment may be formed from a single piece of fabric. One or more portions of the undergarment may be formed from multiple pieces of fabric. One or more portions of the undergarment may be any color or combination of colors. One or more portions of the undergarment may have a pattern (e.g., striped, checkered, seasonal, tie dye, flowered, and the like). Different colors or patterns may be used in different areas of the undergarment. For example, the torso portion may be one color, and the bottom portion may be a different color. One or more portions of the undergarment may include one or more textured portions on the inner surface (e.g., against the user), on the outer surface (e.g., away from the user), or both. The textured portion may be for decoration, may hold an outer garment in place on the user, may hold the traditional garment top in place (e.g., resisting movement through increased friction between materials), or combination thereof.

The torso portion may contact or cover at least a portion of a wearer's torso. For example, the torso portion may contact or cover a protruding belly portion or skin, at least a part of a back of the wearer, sides of a wearer, or a combination thereof. The torso portion may be generally tubular or cylindrical. The torso portion may be fabricated from the same material, color, pattern, etc. as the bottom portion, or a different material, color, pattern, etc. The torso portion may be integrally formed with the bottom portion, or may be attached thereto via stitching or other fasteners. The torso portion may include one or more openings (i.e., torso openings) and one or more flaps facilitating putting on and taking off the undergarment. The one or more flaps may be any provision, cut, slit, opening, notch, protrusion, etc. that may make putting on and taking off the undergarment easier. The one or more flaps may have a length that is less than about half of a height of the undergarment. The one or more flaps may face the front of a wearer (i.e., the belly of the wearer), a back of user (i.e., the back of the wearer), a side of a user, or a combination thereof. The one or more flaps may include one or more fasteners for securing the torso portion, the undergarment, or both around the torso of a wear. The one or more fasteners may be any fastener that may preform the aforementioned functions. For example, the one or more fasteners may be one or more buttons, snaps, hook and eyes (i.e., Velcro®), clasps, zippers, etc. Such fasteners may be located along one or more of the front, back side or an alternative location along the undergarment. The figures herein show a hook and eye arrangement along the front of the undergarment but it is just as possible that the fastener may be located along the back of the undergarment.

The torso portion may include one or more compression features. The one or more compression features may lift and/or support the weight of a wearer's belly area; may provide compression to a wearer's belly area to encourage shrinkage of skin; may provide compression to the torso, back, sides, hips, buttocks, or thighs of the wearer; or a combination thereof. The one or more compression features may provide a smoother appearance to skin in the torso, belly, hips, buttocks, thighs of the wearer. The one or more compression features may function to reduce the mass of loose skin, tissue, and/or muscle. The one or more compression features may discourage swelling; may assist in organizing collagen formation; may provide support to one or more portions of the anatomy of the wearer, or a combination thereof. The one or more compression features may facilitate a faster return to a woman's pre-pregnancy figure. The one or more compression features may provide for the torso portion, the undergarment, or both to stretch and snuggly or compressably fit around a wearer's torso and/or bottom. For example, the circumference of the torso portion of the undergarment may be from about 12 inches to about 40 inches. When stretched horizontally (i.e., when the undergarment is worn), the one or more compression features may increase the circumference of the undergarment by about 1.1× or more, about 1.25× or more, about 1.5× or more, about 2× or more, or even about 3× or more. The circumference may increase by about 10× or less, about 7× or less, about 5× or less, about 4× or less, or even about 3.5× or less. The one or more compression features may include one or more compression materials, one or more compression bands, one or more compression strips, or a combination thereof.

The one or more compression materials may be any feature(s) or material(s) that may be used to perform any of the functions recited herein. The one or more compression materials may help to hold the undergarment in place while the wearer is wearing it. The one or more compression materials may be any stretchable, compressive, elastic, resilient, or memory fabric or material, or a combination thereof. The one or more compression materials may form, or may be in included in one or more portion of the undergarment, such as the torso portion, the bottom portion, or both. The one or more compression materials may be integrated into an outer surface of the torso portion, the inner surface of the torso portion, or both. The one or more compression materials may also be incorporated into one or more portions of the bottom portion of the undergarment.

The one or more compression bands may be any feature(s) or material(s) that may perform any one of the aforementioned functions. The one or more compression bands may help to hold the undergarment in place while the wearer is wearing it. The one or more compression bands may have a circumference that will accommodate a person of any size range. For example, the one or more compression bands may accommodate a pregnant woman and/or a woman post-pregnancy seeking to return to her pre-pregnancy measurements. The one or more compression bands may accommodate a person who is not pregnant or was not pregnant recently. The one or more compression bands may be any elastic, ribbing, anti-slip or tacky substance (e.g., silicone band and/or lining), or other feature or combination of features. The one or more compression bands may be located anywhere on the undergarment. For example, the one or more compression bands may form, or may be in included in one or more portion of the undergarment, such as the torso portion, the bottom portion, or both. More specifically, the one or more compression bands may be located at the top of the torso portion, at the top of the bottom portion, at the leg openings, along a portion or all of one or more sides of the undergarment, etc. The one or more compression bands may circumscribe at least a portion of the torso portion, the torso opening, the bottom portion, the leg openings, or a combination thereof. The one or more compression bands may extend about 360 degrees or less around the torso opening. Preferably, the one or more compression bands extend less than 360 degrees around the torso opening. More preferably, the one or more compression bands extend between opposing sides of the flaps. In other words, in some configurations, the one or more compression bands are not disposed across the flap; however, in other configurations, the one or more compression bands ma be disposed across the flap.

The one or more compression strips may be any feature(s) or material(s) that may perform any one of the functions recited herein. The one or more compression strips may help to hold the undergarment in place while the wearer is wearing it. The one or more compression strips may form, or may be included in one or more portion of the undergarment, such as the torso portion, the bottom portion, or both. The one or more compression strips may be boning. The one or more compression strips may function to maintain the structure and shape of the undergarment and to prevent sagging. The one or more compression strips may be attached to the undergarment. For example, the one or more compression strips may be sewn or stitched into the undergarment. Preferably, the one or more compression strips are sewn or incorporated into a lining or encased at one or more openings or provisions. The one or more compression strips may be fabricated from any suitable material for performing the functions recited herein. For example, the one or more compression strips may be one or more rigid rods or strips of a resilient metal, plastic, elastic, memory material, etc. The one or more compression strips may be arranged in any manor. For example, the one or more compression strips may be arranged on the undergarment, torso portion, bottom portion either vertically, horizontally, may crisscross, intersect, touch, etc. or a combination thereof. The one or more compression strips may be any shape. For example, the one or more compression strips may be linear, circular, irregular, curved, arcuate, etc. The one or more compression strips may be of any length or height. Preferably, the one or more compression strips have a length or height that is generally less than an overall height of the undergarment so that the undergarment provides compression at the belly or back regions of the anatomy of the wearer. More preferably, the one or more compression strips may have a length that is about ⅓ of the overall height of the undergarment so that the undergarment can be easily and comfortably worn. The one or more compression strips may be of any width. The one or more compression strips may have a thickness that is generally greater than the thickness of the portions of the undergarment, less than the thickness of the portions of the undergarment, approximately equal to the thickness of the portions of the undergarment, or combination thereof. The one or more compression strips may be located on the inside surface of the undergarment (i.e., in contact with the skin); on the outside surface of the undergarment; or both. The thickness of the one or more compression strips and location of the one or more compression strips (e.g., on the inside surface, outside surface, or both) may be dependent upon the amount of support desired for the person's body. The one or more compression strips may comprise one or more compression strips, two or more compression strips, three or more, four or more, five or more, or even eight or more compression strips.

The bottom portion may provide full or partial coverage of the person's back, sides, buttocks, hips, legs, or combination thereof. The bottom portion may provide slight compression of a person's back, sides, buttocks, hips, legs, or combination thereof. The bottom portion may be integrally formed with the torso portion, or may be attached thereto. The bottom portion can resemble any underwear (i.e., briefs, boy shorts, bikini, thong, low-rise, support style, full or high cut, etc.), and/or can be incorporated into an integrally formed or attached stockings, such that the undergarment comprises a pair of pantyhose. The bottom portion may be generally tubular or cylindrical. The bottom portion may be seamless. The bottom portion may include seams. The bottom portion may include one or more leg openings. Each of the leg openings may encircle at least a portion of the person's leg or thigh.

The bottom portion may include one or more medicament areas. The one or more medicament areas may be any feature(s) functioning, providing, promoting, and/or facilitating wound or scar management and/or treatment. The one or more medicament areas can be located virtually anywhere on the undergarment (i.e., the bottom portion, the torso portion, etc.) to treat any scar or wound anywhere on the anatomy where the undergarment may cover, reach, etc. The one or more medicament areas can extend from the undergarment to heal, treat, etc. a scar or wound not covered or in contact with the undergarment. The one or more medicament areas may treat post-pregnancy scars, wounds, injuries, etc. The one or more medicament areas may treat post-cesarean scars. The one or more medicament areas may provide protection against bacteria, viruses, fungi, etc. The one or more medicament areas may include an antimicrobial agent. The one or more medicament areas may be or include silver, silver sulfadiazine, silver chloride, sliver nitrate, silver foil, or a combination thereof. The one or more medicament areas may provide a sustained release of positively charged sliver ions at the wound surface, which can promote wound healing and decrease infection by killing bacteria. The one or more medicament areas may comprise one or more of: a silicone gel sheeting, a silica derived synthetic polymer, silicone gel, pads and fluids; elastomers (another silicone polymer); or other moisture retaining/applying substances; silicone or other dressings and substances impregnated with wound healing adjuncts such as, but not limited to aloe, moisturizers or antibiotics; fabric coated with scar management mediums; foams or any other derived substances purported to assist in wound healing.

The one or more medicament areas may be one or more panels, inserts, dressings, applicators, gels, pads, fluids, etc. The one or more medicaments may be varied sizes, thicknesses, densities so that the amount of medicament can be customized for the wearer and would be dependent on a list of variables including the size of the wound or scar, the person's history of wound healing, the length of time since the procedure, the person's body habits, the time of day (night versus day comfort), and the ambient temperature (lighter or heavier silicone layer) to name a few. The one or more medicament areas may be attached to the inside surface of any portion of the undergarment (e.g., the torso portion, the bottom portion, or both). The one or more medicament areas may be permanent attached to the undergarment, or, preferably, removably attached to the undergarment so that the medicament areas can be replaced, cleaned, changed, etc. The one or more medicaments can be attached to one or more portions of the undergarment via any suitable fastener. For example, the one medicament area can be attached to the undergarment via one or more buttons, snaps, hook and eyes (i.e., Velcro), clasps, zippers, etc. The one or more medicament areas can be integrally formed with the undergarment, the torso portion, the bottom portion, or a combination thereof.

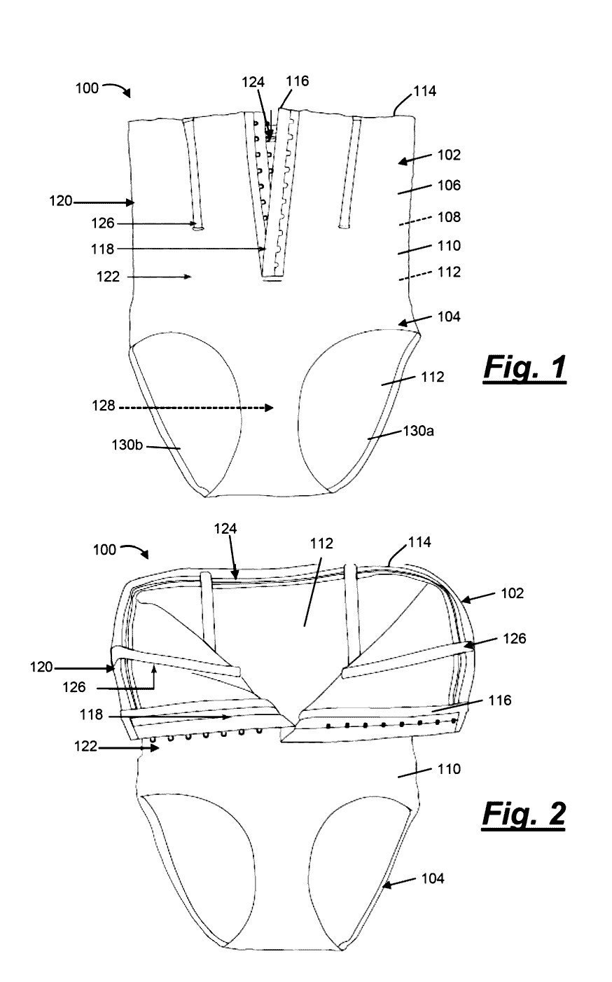

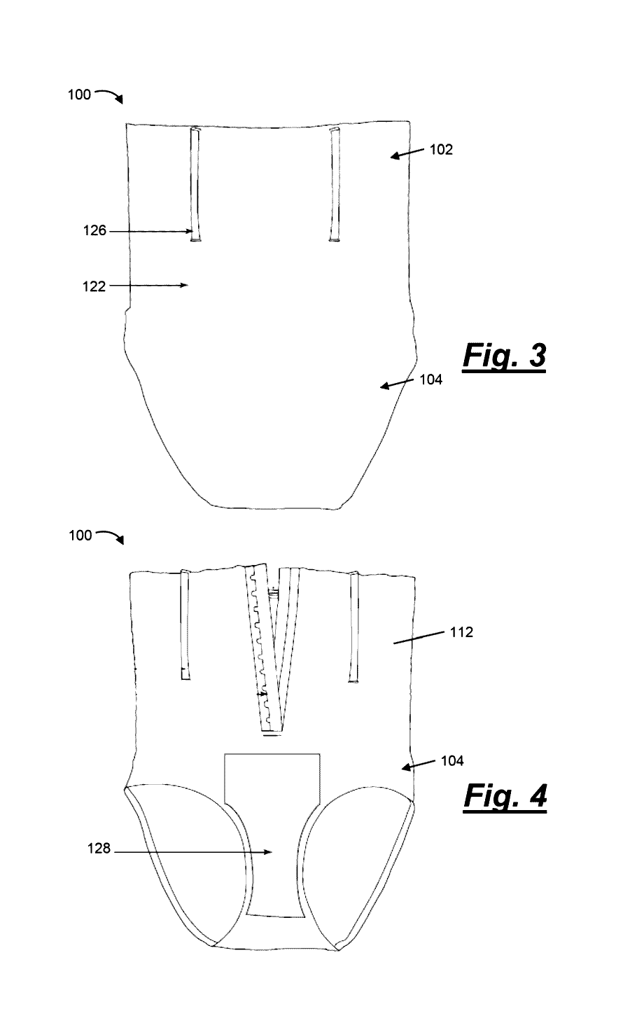

FIGS. 1 and 2 illustrate front views of an undergarment 100, which generally includes a torso portion 102 and a bottom portion 104. The torso portion 102 and the bottom portion 104 may provide various supporting functions to the wearer's body, including coverage, compression, shaping, lifting, and smoothing of a wearer's torso and bottom, respectively. The torso portion 102 and the bottom portion 104 each include a first side 106 and an opposing second side 108. The second side 108 may be best shown in FIG. 3. Each side 106, 108 includes an outer surface 110 and an opposing inner surface 112. The torso portion 102 includes a torso opening 114 fitting around the wearer's torso; a flap 116 comprising a fastener 118 for comfortably securing the undergarment to the wearer's torso; and compression features 120 providing at least some of the aforementioned supporting functions. The compression features 120 include one or more compression materials 122, one or more compression bands 124, and one or more compression strips 126. The bottom portion 104 includes a medicament area 128, which may be best shown in FIG. 4 and leg openings 130a, 130b. The medicament area 128 may be used to treat various wounds, scars, infections, cesarean incisions, etc.

FIG. 3 illustrates a back view of the torso portion 102 and the bottom portion 104 of the undergarment 100. The torso portion 102 includes one or more compression materials 122 and one or more compression strips 126.

FIG. 4 illustrates the inner surface 112 of he undergarment 100. The bottom portion 104 includes a medicament area 128.

Any, all, or a combination of the features disclosed herein is contemplated to be within the scope of the teachings. The above described preferred embodiments are intended to illustrate the principles of the invention, but not to limit its scope. Other embodiments and variations to this preferred embodiment will be apparent to those skilled in the art and may be made without departing from the spirit and scope of the invention as defined in the following claims. While this disclosure is focused primarily on a pregnant woman's body, it is contemplated that this garment may also be used by persons who are not pregnant, men or women, who are looking for a garment to protect traditional garments from specialty skin products, provide a shirt extension, provide support for the lower back and belly, or to provide additional smoothing, compression and shaping of hips, thighs, and buttocks. Furthermore, the words man, woman, user, person, wearer, and variations of these words herein are meant to be synonymous, and use of any of these words does not exclude the others unless otherwise stated.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the of a range in terms of at “x′ parts by weight of the resulting polymeric blend composition” also contemplates a teaching of ranges of same recited amount of “x” in percent by weight of the resulting polymeric blend composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30,” inclusive of at least the specified endpoints.

The disclosures of all articles and references, including patent applications and publications, are incorporated by reference for all purposes. The term “consisting essentially of to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist of, or consist essentially of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

It is understood that the above description is intended to be illustrative and not restrictive. Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

What is claimed is:

1) An undergarment, comprising:
a torso portion to be worn around a torso of a wearer, the torso portion comprising:
i. one or more compression features; and
a bottom portion comprising:
i. a medicament area
wherein the one or more compression features provide compression against at least a portion of the torso of the wearer, and
wherein the medicament area interacts with a scar and/or wound and facilities healing thereof.

2) The undergarment of claim 1, wherein the one or ore compression features includes one or more compression materials,
wherein the one or more compression materials cover at least a part of a belly and at least a part of a back of the wearer.

3) The undergarment of claim 1, wherein the one or more compression features includes a compression band, wherein the compression band is disposed at a torso opening of the torso portion.

4) The undergarment of claim 3, wherein the compression band extends less than 360 degrees around the torso opening.

5) The undergarment of claim 1, wherein the one or more compression features includes a plurality of compression strips, wherein each of the compression strips are extend vertically from an opening of the torso portion.

6) The undergarment of claim 5, wherein each of the compression strips has a length that is about ⅓ of a height of the undergarment.

7) The undergarment of claim 1, wherein the medicament area includes at least some silver,
wherein the medicament area interacts with the scar and/or wound located at or near a cesarean incision site.

8) The undergarment of claim 1, wherein the medicament area is removable from the undergarment.

9) The undergarment of claim 1, wherein the one or more compression strips are boning.

10) The undergarment of claim 5, wherein the plurality of compression strips comprises four compression strips.

11) The undergarment of claim 1, wherein the undergarment includes a flap comprising a fastener for securing the undergarment to the torso of the wearer.

12) An undergarment, comprising:
i. a compression band;
ii. a plurality of compression strips; and
iii. a medicament area comprising at least some silver;
wherein the compression band and the plurality of compression strips provide compression against at least a portion of an anatomy of a wearer;
wherein the silver interacts with a scar or wound on the anatomy of the wearer and facilities healing thereof; and
wherein the silver provides for odor elimination and/or reduction.

13) The undergarment of claim 12, wherein the compression band extends near a torso opening of the undergarment, and extends less than about 360 degrees around the torso opening.

14) The undergarment of claim 12, wherein each of the compression strips has a length that is about ⅓ of a height of the undergarment.

15) The undergarment of claim 14, wherein the undergarment comprises a flap including a fastener for securing the undergarment to the torso of the wearer, and wherein a length of the flap is greater than the length of at least one of the compression strips.

16) The undergarment of claim 12, wherein the silver interacts with the scar or wound at a cesarean incision site and facilitates healing thereof.

17) An undergarment, comprising:
a torso portion to be worn around a torso of a wearer, the torso portion comprising:
i. a compression material;
ii. a compression band; and
iii. a plurality of compression strips;
a bottom portion to be worn around at least a portion of a buttocks of the wearer, the bottom portion comprising:
ii. a medicament area;
wherein the compression material, the compression band, and the plurality of compression strips provide compression against at least a portion of the torso of the wearer; and
wherein the medicament area includes at least some silver that interacts with a scar or wound at a cesarean incision site and facilities healing thereof and reduces odor.

18) The undergarment of claim 17, wherein the torso portion covers at least a part of a protruding belly portion or skin and at least a part of a back of the wearer, and
wherein the bottom portion covers at least a portion of the wearer's buttocks and thighs.

19) The undergarment of claim 18, wherein the medicament area is removable from the bottom portion of the undergarment.

20) The undergarment of claim 18, wherein the plurality of compression strips are four compression strips, and
wherein the compression strips are boning.
